(19) United States
(12) Patent Application Publication
(10) Pub. No.: US 2025/0281240 A1
(43) Pub. Date: Sep. 11, 2025

(54) METHOD FOR HELPING A USER IN THE PLANIFICATION OF A TREATMENT TO BE DELIVERED VIA A PROBE

(71) Applicant: AVATAR MEDICAL, Paris (FR)

(72) Inventors: Mohamed EL BEHEIRY, Paris (FR); Elodie BRIENT-LITZLER, Paris (FR); John ROSS, Paris (FR); Xavier WARTELLE, Paris (FR)

(73) Assignee: AVATAR MEDICAL, Paris (FR)

(21) Appl. No.: 19/214,292

(22) Filed: May 21, 2025

Related U.S. Application Data
(63) Continuation of application No. PCT/EP2023/082870, filed on Nov. 23, 2023.

(30) Foreign Application Priority Data
Nov. 25, 2022 (EP) 22306745.5

(51) Int. Cl. A61B 34/10; G06F 3/04815; G06F 3/04845; G06T 19/00

(52) U.S. Cl. CPC A61B 34/10; G06F 3/04815; G06F 3/04845; G06T 19/00; A61B 2034/102; A61B 2034/105; A61B 2034/107; G06T 2200/24; G06T 2210/41

(57) ABSTRACT

A computer-implemented method for helping a user in the planification of a treatment to be delivered via a probe to an effective target region of a body of a patient, the treatment being a radiotherapy, an ablation therapy, a laser-based or an ultrasound-based treatment, by a user graphical interface including a controller configured to control the interaction between the user and the user graphical interface. A device is configured to implement the method and to a computer program product including instructions which, when the program is executed by a computer, cause the computer to carry out the method.

### REFERENCE TO RELATED APPLICATIONS

This application is a bypass continuation of International Patent Application No. PCT/EP2023/082870, filed on Nov. 23, 2023, which claims the benefit of 22306745.5, filed Nov. 25, 2022, each of which is incorporated by reference herein in its entirety.

### FIELD

The present invention relates to a computer-implemented method for helping a user in the planification of a treatment to be delivered via a probe to a target region of a body of a patient, said treatment being a radiotherapy, an ablation therapy, a laser-based or an ultrasound-based treatment.

### BACKGROUND

It is known to use a series of images of a patient's body area taken before an intervention. This series of images, called “planning data” or “planning images”, is used by doctors to prepare the intervention, in particular by visualizing the body area on which they must intervene and by locating the diseased tissues as well as determine areas and tissues that are to be preserved.

In the case of medical interventions where a treatment has to be delivered to a target region of a body patient by the doctors via a probe, precise knowledge of how the probe will interact with the target region is needed, so as to maximize the effects of the treatment while minimizing the risks this treatment may bring.

The invention aims at improving the situation.

### SUMMARY

More precisely, the invention aims at allowing to gain insight on the effects of interaction of a probe delivering a treatment dose to a patient during the planification phase thanks to an interactive method.

This invention thus relates to a computer-implemented method for helping a user in the planification of a treatment to be delivered via a probe to an effective target region of a body of a patient, said treatment being a radiotherapy, an ablation therapy, a laser-based or an ultrasound-based treatment, by means of a user graphical interface comprising a controller configured to control the interaction between the user and the user graphical interface, said method comprising:
displaying in the user graphical interface a three-dimensional scene comprising a body 3D model of at least a portion of a body of the patient and a probe 3D representation of at least a portion of a probe; said probe 3D representation being interactively controlled by the controller; wherein the three-dimensional scene has a corresponding scene coordinate system attached thereto;
computing a current position and current orientation of the probe 3D representation in the three-dimensional scene coordinate system based on a current position of the mechanical controller in a predetermined user coordinate system;
computing an initial treatment 3D distribution corresponding to said current position and current orientation;
displaying a representation of said initial treatment 3D distribution overlaid with said body 3D model.

Thanks to the invention, it is possible to associate a treatment 3D distribution to a position of the controller in the predetermined user coordinate system and to visualize how the treatment distribution is localized with respect to the body 3D model.

According to other advantageous aspects of the invention, the method comprises one or more of the features described in the following embodiments, taken alone or in any possible combination.

In some embodiments, the method further comprises cropping said representation so as to display a 2D cross-section of said initial treatment 3D distribution. Notably, the cropping operation is performed by the method in response to an instruction provided by the user via said controller.

In some embodiments, the initial treatment 3D distribution comprises a set of treatment voxels, each voxel in said set of treatment voxels being associated to a voxel set of parameters.

In some embodiments, for each voxel in the set of treatment voxels, the associated voxel set of parameters comprises an intensity value.

In some embodiments, the three-dimensional scene further comprises a target region.

In some embodiments, the three-dimensional scene further comprises a region of interest.

In some embodiments, the method further comprises identifying target voxels in the set of treatment voxels that are located inside the target region if the three-dimensional scene further comprises a target region, and identifying interest voxels in the set of treatment voxels that are located inside the region of interest if the three-dimensional scene further comprises a region of interest.

In some embodiments, the method further comprises computing a first index of superimposition based on the intensity values of the identified target voxels and displaying said first index of superimposition. Thanks to the first index of superimposition, it is possible to have insight on how the treatment 3D distribution overlaps or fill the target region.

In some embodiments, the method further comprises computing a second index of superimposition based on the intensity values of the identified interest voxels and displaying said second index of superimposition. Thanks to the first index of superimposition, it is possible to have insight on how the treatment 3D distribution overlaps or fill the region of interest.

In some embodiments, the first index of superimposition is a sum of the intensity values of the identified target voxels.

In some embodiments, the second index of superimposition is a sum of the intensity values of the identified interest voxels.

In some embodiments, the first index of superimposition is a first weighted sum of the intensity values of the identified target voxels, the first weighted sum being parameterized by weights, each weight being associated with one among the identified target voxels and being representative of a absorption coefficient of the associated identified target voxel.

In some embodiments, the second index of superimposition is a second weighted sum of the intensity values of the identified interest voxels, said second weighted sum being parameterized by weights, each weight being associated with one among the identified interest voxels and being representative of an absorption coefficient of the associated identified interest voxel.

In some embodiments, the method further comprises:
receiving another current position of the controller in the predetermined user coordinate system,
computing a corrective position and a corrective orientation of the probe 3D representation in the three-dimensional scene coordinate system based on said another current position,
computing a corrective treatment 3D distribution corresponding to the corrective position and corrective orientation,
displaying a representation of the corrective treatment 3D distribution overlaid with said body 3D model.

Therefore, it is possible to explore how a change in the probe position and orientation impact the localization of the treatment 3D distribution with respect to the body 3D model.

In some embodiments, when the three-dimensional scene further comprises a target region, the corrective treatment 3D distribution comprises a set of corrective treatment voxels, and the method further comprises:
identifying corrective target voxels in the set of corrective treatment voxels that are located inside the target region,
computing a first corrective index of superimposition based on the intensity values of the identified corrective target voxels,
displaying the first corrective index of superimposition.

The first corrective index of superimposition helps getting insight on how the corrective position and the corrective orientation of the probe 3D representation has improved or degraded the overlapping or the filling of the target region by the treatment 3D distribution.

In some embodiments, when the three-dimensional scene further comprises a region of interest, the corrective treatment 3D distribution comprises a set of corrective treatment voxels, and the method further comprises:
identifying corrective interest voxels in the set of corrective treatment voxels that are located inside the region of interest,
computing a second corrective index of superimposition based on the intensity values of the identified corrective interest voxels,
displaying the second corrective index of superimposition.

The first corrective index of superimposition helps getting insight on how the corrective position and the corrective orientation of the probe 3D representation has improved or degraded the overlapping or the filling of the region of interest by the treatment 3D distribution.

In some embodiments, the target region is a tumor.

In some embodiments, the region of interest comprises an organ at risk.

In some embodiment, the method further comprises identifying, when the second corrective index of superimposition is higher than a threshold value, displaying an alert message or modifying the display of the region of interest to alert the user.

Indeed, when the region of interest in an organ at risk, the region of interest has to be avoided by the treatment 3D distribution. Therefore, a control of the value of the second index of superimposition helps adjusting or validating a given position and orientation of the probe 3D representation.

In some embodiments, the threshold value is received through the user graphical interface.

In some embodiments, the method further comprises computing angular coordinates representative of an orientation of the probe 3D representation with respect to a reference orientation of said body 3D model.

In some embodiments, the method further comprises displaying said angular coordinates and recording said angular coordinates into an external memory.

In some embodiments, the method further comprises transferring the computed angular coordinates to a mechanical system, mounting the probe, to be used to assist the user in the positioning of the probe during the treatment.

Indeed, when an optimal position and orientation of the probe 3D representation is found, for instance maximizing the first index of superimposition, it may be useful to know and record to which real geometric configuration of the real probe in the real intervention referential this optimal position and orientation of the probe 3D representation corresponds, so as to reproduce it when effectively carrying out the intervention.

In some embodiments, the three-dimensional scene further comprises at least one other probe 3D representation of a portion of another probe, the at least one other probe 3D representation being interactively controlled by the controller and independently from the probe 3D representation, and the method further comprises:
optionally receiving a selection of a subset among the probe 3D representation and the at least one other probe 3D representation in an active state,
computing a complete treatment 3D distribution based on: a) either contributions of the probe 3D representation and the at least one other probe 3D representation, or b) contributions of the received selection,
displaying a representation of the complete treatment 3D distribution overlaid with said body 3D model.

Indeed, in some cases, one probe is not sufficient to deliver enough treatment to the effective target region. Therefore, advantageously, it is possible to interact with a plurality of probe 3D representations and getting insight on their overall effect.

In some embodiments, the complete treatment 3D distribution comprises a complete set of treatment voxels, each voxel in said complete set of treatment voxels being associated to a voxel set of parameters, each voxel set of parameters comprising an intensity value, and the method further comprising:
identifying first complete voxels in the current set of treatment voxels that are located inside the target region,
computing a first complete index of superimposition based on the intensity values of the identified first complete voxels,
displaying said first complete index of superimposition.

The first complete index of superimposition helps getting insight on the effect of all the probes on delivering the treatment to the target region.

In some embodiments, the complete treatment 3D distribution comprises a complete set of treatment voxels, each voxel in said complete set of treatment voxels being associated to a voxel set of parameters, each voxel set of parameters comprising an intensity value, and the method further comprising:
identifying second complete voxels in the complete set of treatment voxels that are located inside the region of interest,
computing a second complete index of superimposition based on the intensity values of the identified second complete voxels,
displaying said second complete index of superimposition.

In some embodiments, the body 3D model is obtained based on a plurality of images comprising said at least portion of a body of the patient, said images being CT scan images, MRI images, or cone beam CT images.

In some embodiments, the user graphical interface comprises a stereoscopic displaying unit.

In some embodiments, the controller is one among a mouse, a 3D controller connected to a virtual reality headset.

In some embodiments, the controller is a home-made controller having a probe-like shape.

Another aspect of the invention pertains to a device comprising a processor, a user graphical interface comprising a controller configured to control the interaction between a user and the user graphical interface, said processor being configured to carry out the method according to any of the previous embodiments.

Another aspect of the invention pertains to a computer program product comprising instructions which, when the program is executed by a computer, cause the computer to carry out the method according to any of the previous embodiments.

The present disclosure further pertains to a non-transitory program storage device, readable by a computer, tangibly embodying a program of instructions executable by the computer to perform a method for helping a user in the planification of a treatment to be delivered via a probe, compliant with the present disclosure.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM).

### BRIEF DESCRIPTION OF THE DRAWINGS

FIG. 1 is a schematic representation of a device configured to implement the method according to embodiments of the present invention.

FIG. 2 is a schematic representation of a processing unit of the device of FIG. 1.

FIG. 3 is an example of a three-dimensional scene that can be visualized on a display unit of the device of FIG. 1, comprising a body 3D model, a probe 3D representation and a treatment 3D distribution.

FIG. 4 is an example of flow chart of the method for helping a user in the planification of a treatment to be delivered via a probe to an effective target region of a body of a patient.

FIG. 5 is a first example of a treatment 2D cross section, for a given 2D plane.

FIG. 6 is a second example of a treatment 2D cross section, for a 2D plane different from the given 2D plane of FIG. 5.

FIG. 7 is an example of a three-dimensional scene that can be visualized on a display unit of the device of FIG. 1, the three-dimensional scene comprising a target region.

FIG. 8 is an example of a three-dimensional scene that can be visualized on a display unit of the device of FIG. 1, the three-dimensional scene comprising a target region, a region of interest and a second region of interest.

### DETAILED DESCRIPTION

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present.

The terms “adapted”, “augmented” and “configured” are used in the present disclosure as broadly encompassing initial configuration, later adaptation or complementation of the present device, or any combination thereof alike, whether effected through material or software means (including firmware).

The term “processing unit” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

It is assumed here that a patient is about to undergo a medical intervention. During this intervention, a portion of his body will be treated by doctors via at least one probe. For instance, in the case of a radiotherapy intervention, a beam will illuminate the portion of the patient's body. In the case of an ablation therapy, the probe will deliver, notably transfer, energy to heat or cool the portion of the patient's body. In the case of an ultrasound-based treatment, ultrasound waves will impinge on the portion of the patient's body.

The invention relates to a computer-implemented method for helping a user 4 such as a physician to plan a medical intervention as described above.

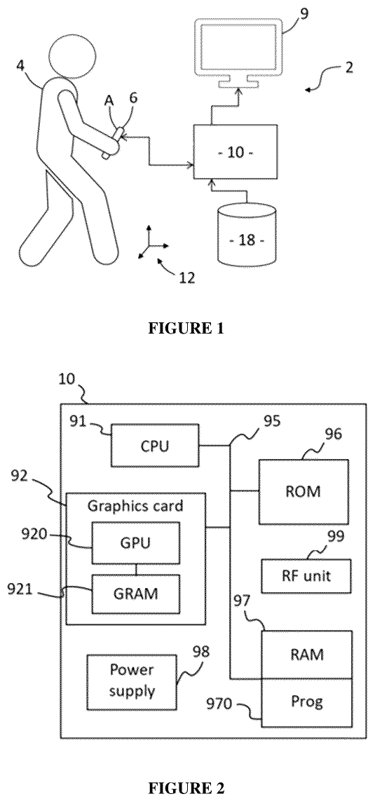

This method may be implemented by a device as illustrated in FIG. 1.

FIG. 1 is a schematic representation of a device configured to implement embodiments of a method for helping a user in the planification of a treatment to be delivered via a probe to a target region of a body of a patient.

The device is advantageously an apparatus, or a physical part of an apparatus, designed, configured and/or adapted for performing the mentioned functions and produce the mentioned effects or results. In alternative implementations, the device is embodied as a set of apparatus or physical parts of apparatus, whether grouped in a same machine or in different, possibly remote, machines. The device may for example have functions distributed over a cloud infrastructure and be available to users as a cloud-based service, or have remote functions accessible through an API (“application programming interface”).

The device includes a user graphical interface 2, comprising a controller 6 (also called joystick) for interacting with a three-dimensional scene, that will be described more in details below, a display unit 9 for displaying the three-dimensional scene, and a processing unit 10 for processing data received from the controller 6 and for controlling the display unit 9.

### Controller 6

The controller 6 is configured to acquire a current position of a predetermined user point A, of a user 4, in a predetermined user coordinate system 12. Notably, the controller 6 may have an elongated shape (see example of FIG. 1) and being configured to track its own position and orientation in a 3D space (user coordinate system 12).

The predetermined user coordinate system 12 is, for instance, fixed with respect to the user's environment. The predetermined user coordinate system 12 uses one or more numbers, or coordinates, to uniquely determine the position of points or other geometric elements in the user's environment.

The controller 6 may include a handheld motion tracking sensor, thereby allowing the user 4 to interact with the three-dimensional scene, represented in the user graphical interface 2, using hand gestures. In this case, the user point A may be a point of the motion tracking sensor. More in details, the controller 6 may be equipped with sensors (like accelerometers and gyroscopes) that enable the method to track its position and orientation in 3D space. The method may also relay on the use of external sensors or cameras to enhance tracking accuracy of the controller 6.

The controller 6 may be also equipped with haptic feedback mechanisms. These provide tactile sensations to users, such as vibrations or force feedback, enhancing the sense of touch and interaction within the virtual environment and virtual objects represented in the user graphical interface 2.

The controller 6 may also include other devices, such as a touch screen, a game controller, a mouse and/or a keyboard, thereby further allowing the user 4 to interact with the three-dimensional scene as will be explained later.

The controller 6 may further include buttons, triggers, switches and/or other input mechanisms configured to allow the user 4 to interact with the three-dimensional scene. Such buttons and/or switches may be included in the handheld motion tracking sensor(s) or may be included in a separate device of the controller 6, such as the touch screen, game controller, mouse and/or keyboard mentioned above.

The controller 6 may include or may be a 3D controller connected to a virtual reality headset. In this case, the user graphical interface 2 and the display unit 9 may be comprised in the virtual reality headset.

The controller may be a home-made controller having a probe-like shape.

Preferably, the controller 6 is configured to allow the user 4 to input (for example through said buttons and/or switches) specific instructions, such as an object display instruction, an object shifting instruction or a transformation instruction. Such instructions advantageously allow the user 4 to add and/or manipulate 3D objects represented in the three-dimensional scene, as will be described below.

The controller 6 may also be configured to allow the user to manipulate (e.g., to rotate and/or to zoom in on or out of) 3D objects in the three-dimensional scene, and/or to change a direction along which the user 4 views the three-dimensional scene displayed by the display unit 9.

Alternatively, or in addition, the controller 6 includes, or the display unit 9 is configured to visualize, virtual buttons displayed in the three-dimensional scene to allow the user 4 to interact with the three-dimensional scene, and 3D objects in the three-dimensional scene.

### Display Unit 9

As mentioned previously, the display unit 9 is configured to display the three-dimensional scene.

The display unit 9 may include a screen, as shown on FIG. 1.

Alternatively, and advantageously, the display unit 9 is at least part of a virtual reality headset, thereby allowing stereoscopic visualization of the three-dimensional scene. This is particularly advantageous in the field of medicine, since virtual reality visualization, providing an in-depth visualization, allows the user 4 to have a good understanding of actual volumes and precise localization of objects of interest.

### Processing Unit 10

The processing unit 10 is connected to each of the controller 6 and the display unit 9.

The processing unit 10 corresponds, for example, to a workstation, a laptop, a tablet, a smartphone, programmable logical device (e.g., FPGA) for on-board calculation or a head-mounted display (HMD) such as a virtual reality headset.

As shown on FIG. 2, the processing unit 10 may comprise the following elements, connected to each other by a bus 95 of addresses and data that also transports a clock signal:
a microprocessor 91 (or CPU);
a graphics card 92 comprising several Graphical Processing Units (or GPUs) 920 and a Graphical Random Access Memory (GRAM) 921; the GPUs are quite suited to image processing due to their highly parallel structure;
a non-volatile memory of ROM type 96;
a RAM 97;
a power supply 98; and
a radiofrequency unit 99.

Alternatively, the power supply 98 may be external to the processing unit 10.

The controller 6 is, for instance, connected to at least part of the aforementioned modules, for instance through the bus 95.

The display unit 9 is connected to the graphics card 92, for instance through a suitable interface. For instance, a cable can be used for tethered transmissions, or the RF unit 99 can be used for wireless transmissions.

Each of memories 97 and 921 includes registers, which can designate in each of said memories, a memory zone of low capacity (some binary data) as well as a memory zone of large capacity (enabling a whole program to be stored or all or part of the data representative of data calculated or to be displayed). Also, the registers represented for the RAM 97 and the GRAM 921 can be arranged and constituted in any manner. Each of them does not necessarily correspond to adjacent memory locations and can be distributed otherwise (which covers notably the situation in which one register includes several smaller registers).

When switched-on, the microprocessor 91 loads and executes the instructions of the program 970 contained in the RAM 97 to allow operation of the visualization device 2 in the fashion described in the present disclosure.

As will be understood by a skilled person, the presence of the graphics card 92 is not mandatory, and can be replaced with entire CPU processing and/or other implementations.

### Operation

The device is configured to receive as input 18 a three-dimensional model 3 of the portion of the patient's body. This three-dimensional model 3 is referred to in what follows as body 3D model 3.

The body 3D model 3 can have been preliminary computed based on 2D images of the portion of the patient's body, such as CT scan (“Computed Tomography scan”) images, PET (“Positron Emission Tomography”) images, MRI (“Magnetic Resonance Imaging”) images, or cone beam CT images. Alternatively, the device may be configured to receive as input 18 2D images of the portion of the patient's body, and then compute via the processing unit 10, from the received 2D images, the body 3D model.

The device may be further configured to receive at least one target region 11, representing a region of the body 3D model 3 (i.e., a volume in the body 3D model 3) that should be treated with the probe by delivering to this target region 11 a desired amount of energy. This target region (one or more) could for example comprise a solid tumor to be treated or any kind of abnormal tissues that should be removed from the patient. The target region 11 may be obtained by a delineation, manually performed by a physician or an automatically performed by a segmentation algorithm. The result of this delineation may than be used to obtain a 3D representation of said target region 11. target region 11 may be as well associated with information to be received as further input to the device such as a thermal conductivity coefficient, a mean, maximum or minimum energy that could be delivered in this region, and the like.

The device may be further configured to receive at least one region of interest 13, representing a region of the body 3D model 3 (i.e., a volume in the body 3D model 3) that should be preserved as much as possible from the exposure to the energy that will be delivered by the probe during the treatment. This (these) region(s) of interest 13 may represent what is generally called an organ at risk. The region of interest 13 may be obtained by a delineation, manually performed by a physician or an automatically performed by a segmentation algorithm. The result of this delineation may than be used to obtain a 3D representation of said region of interest 13. The region of interest 13 may be as well associated with information to be received as further input to the device such as the radio sensitivity of the tissues in the region of interest, a thermal conductivity coefficient, a maximum energy that could be delivered in this region, and the like. This information may be advantageously used during the calculation of the initial 3D treatment distribution.

The device (notably the processing unit 10) is configured to further receive as input 18 a probe 3D representation 5, being a 3D representation of at least a portion of a probe. In the present invention, the probe refers to a device to deliver a treatment, or more generally medical device, that may be used, for example by a physician, to deliver a treatment to a subject. In the present invention such probe/medical device may be, but not limited to, one of the following: an ablation probe (e.g., cryo ablation probe, radiofrequency ablation probe, microwave ablation probe and the like), a radiotherapy gantry, a sealed radiation source for brachytherapy, an ultrasound therapy probe, and the like.

The processing unit 10 may be as well configured to receive a list of predefined information associated to the probe 3D representation 5, such as the type of energy transferred by the probe (e.g., gamma radiation or x-rays radiation in case of radiotherapy, or ultrasound waves in the case of ultrasound therapy probe, etc.), the range of energies that could be delivered and the like. Advantageously, this information may be used by the processing unit 10 for the simulation of the interaction of the energy with the tissues of the body 3D model, during the calculation of the initial 3D treatment distribution. The processing unit 10 could as well be configured to receive other treatment planning information concerning the treatment such as a mean energy to be transferred or a maximum energy to not overcome to preserve healthy tissues.

As described above, the display unit 9 of the user graphical interface 2 is configured to display, to the user 4, a three-dimensional scene comprising the body 3D model 3 and the probe 3D representation 5 of the at least one portion of a probe. The three-dimensional scene has a corresponding scene coordinate system attached thereto. There is a correspondence between the predetermined user coordinate system 12 and the corresponding scene coordinate system.

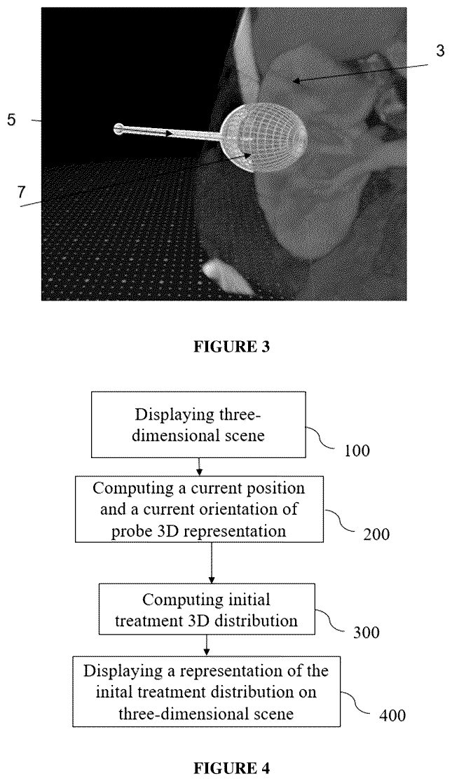

FIG. 3 is an example of a three-dimensional scene comprising the body model 3 and the probe 3D representation 5, as displayed on the display unit 9.

In some embodiments, the probe 3D representation 5 is interactively controlled by the controller 6. Indeed, the controller 6 is configured to allow the user to interact with the objects represented in the scene (i.e., virtual environment), such as the probe 3D representation 5 and the body 3D model 3. As a consequence, when the controller 6 is moved in space by the user 4, in the user's environment comprising (i.e., associated to) the predetermined user coordinate system 12, the probe 3D representation 5 moves in the corresponding scene coordinate system. To a position and orientation of the controller 6 in the predetermined user coordinate system 12, corresponds a position and an orientation of the probe 3D representation 5 in the scene coordinate system.

When the user 4 manipulates the controller 6, the position and spatial orientation of the controller 6 is changed. These changes in position and spatial orientation of the controller 6 are continuously monitored by the controller sensors (e.g., IMU embedded in the controller 6) and are translated from the user coordinate system 12 into the scene coordinate system. The person skilled in the art knows different technics allowing transforming real positions of a controller into a virtual environment. As a consequence, the position and orientation of the probe 3D representation 5 change in the scene coordinate system.

For a given position and orientation of the probe 3D representation 5 in the scene coordinate system, a corresponding treatment 3D distribution 7 may be computed. This calculated treatment 3D distribution comprises a set of treatment voxels. The processing unit may be configured to calculate for each voxel of said set of treatment voxels a set of parameters, that will be called in the present description “voxel set of parameters”.

Said initial treatment 3D distribution may be calculated, not only on the based on the given position and orientation of the probe 3D representation 5 in the scene coordinate system, but also on the base of the type of energy delivered/transferred by the probe that is simulated. Indeed, as explained above, the probe to be simulated in the present invention may be chosen from a list of multiple probes/medical devices and be configured to deliver a treatment based on the delivery of different type of energy. Depending in the type of energy to be transferred to the tissues, the physical interactions between the “carrier” of this energy (i.e., radiation, microwaves, ultrasounds, etc.) and the tissues change and so it changes the 3D distribution of the energy (i.e., treatment 3D distribution) in the body 3D volume. Advantageously, the information on the type of probe (i.e., on the type of energy delivered by the probe) allows the processing unit 10 to calculate the initial 3D distribution on the base of the simulation of the corresponding physical interactions. Furthermore, the other information concerning the treatment to be delivered by the probe may be taken into account during the calculation such as the list of predefined information associated to the probe 3D representation 5, the information associated with the target region 11 and/or the information associated with the region of interest 13.

One of the parameters (of said voxel set of parameters) that could be calculated for each voxel is an intensity value representative of an amount of energy transferred by the probe to the voxel. In the case of radiation therapy or brachytherapy, other parameters that may be calculated such as the dose delivered to the tissue associated to the voxel, calculated in Gray, or the effective dose, calculated in Sieverts, if the potential biological effects of ionization radiation and the radio sensitivity of the tissues is taken into account. In the case of cryo ablation, laser ablation and the like, a parameter calculated may be the temperature, or the maximum temperature, that will be reached in the voxel during the treatment.

For instance, in the case of a radiotherapy intervention, the probe (e.g., a gantry) is configured to deliver a beam that will illuminate (i.e., irradiate) the portion of the patient's body, notably the totality of the target region 11 while trying to avoid as much as possible the region(s) of interest 13 (i.e., organs at risk). The corresponding initial treatment 3D distribution 7 then corresponds to a set of voxels, the voxels being referred to as treatment voxels. The set of treatment voxels is representative of the spatial distribution of the energy that is transferred by the beam delivered by the probe in a given position and orientation. Thus, the set of treatment voxels covers a 3D area (i.e., a volume) in the scene coordinate space. The 3D area is computed at least based on the probe real characteristics (i.e., list of predefined information associated to the probe 3D representation 5) and on the probe 3D representation position and orientation in the scene coordinate space. In addition, each treatment voxel in the set of treatment voxels is associated to a voxel set of parameters. For instance, for a given treatment voxel, the voxel set of parameters comprises an intensity value, representing the dose that would be delivered by the radiation beam to the tissue associated to the voxel. Each intensity value is computed based on the probe real characteristics (i.e., list of predefined information associated to the probe 3D representation 5) and on the probe 3D representation position and orientation in the scene coordinate space. The treatment planning information may be used as additional information for a more accurate calculation of the intensity values.

Similarly, in the case of an ablation therapy, the probe delivers/transfers energy that will heat or cool the portion of the patient's body. The initial treatment 3D distribution 7 corresponding to the delivered energy can be represented by a set of voxels. In other words, the initial treatment 3D distribution 7 represents all the voxels of the body 3D model 3 that will have a change in temperature because they will be affected by the energy transfer, either by improving the energy of the biological system when heat is transferred (i.e., absorbed) or by reducing the energy of the biological system when the heat is extracted (i.e., cooling down).

In the case of an ultrasound-based treatment, the probe emits ultrasound waves that will impinge on the portion of the patient's body. The treatment 3D distribution 7 corresponding to the emitted ultrasound waves can be represented by a set of voxels. In other words, in the case of ultrasounds, the treatment 3D distribution 7 comprises all the voxels of the body 3D model 3 that are reached by the ultrasound waves emitted by the probe. The ultrasound waves may be used in physical therapy to apply controlled amounts of energy to targeted tissues for therapeutic benefits. This can include promoting tissue healing, reducing inflammation, and providing pain relief. The energy is delivered through the skin using a specialized ultrasound transducer (i.e., ultrasound probe). As consequence, the treatment 3D distribution 7 provides a representation of the energy that would be transferred by the ultrasound waves to the tissues when the ultrasound probe is positioned in a given position and orientation with respect to the body 3D model 3.

In the case of a laser-based treatment, the probe emits a laser that will illuminate the portion of the patient's body. As consequence, in this case, the treatment 3D distribution 7 provides a representation of the energy that would be transferred by the laser to the tissues when the probe is positioned in a given position and orientation with respect to the body 3D model 3.

As described according to all the example hereabove, the treatment 3D distribution 7 provides a representation of the energy that would be transferred to the tissues represented in the body 3D model 3. Therefore, the treatment 3D distribution 7 may be as well called 3D energy distribution.

In some embodiments, after that for one given position and orientation of the probe 3D representation 5 in the scene coordinate system, a corresponding treatment 3D distribution 7 has been computed, a representation of the computed treatment 3D distribution 7 is displayed overlaid on the three-dimensional scene on the display unit 9. An example of such a representation is visible on FIG. 3. Notably in FIG. 3, the probe 3D representation 5 and a visual representation of the treatment 3D distribution 7 are represented in the three-dimensional scene (i.e., virtual environment) at the same time that a partial representation of the body 3D model 3. In this example, the visual representation of the treatment 3D distribution 7 allow to easily identify the distal voxels/external boundaries of the volume of the treatment 3D distribution 7. In this way the user will advantageously have the information at once of the position and direction of the probe 3D representation 5 with respect to the body 3D model 3 and that all voxels comprised in this visual representation of the treatment 3D distribution 7 will be affected by the transferred of energy applied with the probe. Thanks to the overlapping of the visual representation of the treatment 3D distribution 7 with a section of the body 3D model 3, the user is also able to visualize which structures of the patient will be affected during the treatment, when the probe is positioned according to his/her choice.

Furthermore, the section of the body 3D model 3 or the structure comprised in the body 3D model 3 (i.e., vascular system, bones, etc.) to be visualized may be choose during the visualization by the user via the interaction with the controller 6. For example, the user graphical interface 2 may be configured to implement an interactive mean called cropper, allowing to virtually crop the volumes represented in the virtual environment. Finally, the overlay of the probe 3D representation with the body 3D model enables the user to optimize the placement of the probe as relative to specific anatomical structures. For stick-shaped probes, such as notably ablation probes, this overlay may help the user to select an entry point and angle (i.e., spatial orientation) of the probe which might avoid critical organs or vessels.

In one embodiment, the device is configured to receive and store a selection, from the user, of one position and spatial orientation of the probe 3D representation 5 with respect to the body 3D model 3, as additional treatment planning information. This additional treatment planning information may be integrated into a treatment plan, which may comprise a list of at least one treatment action to be performed by the user (i.e., surgeon) with the probe on the patient during the treatment. Each additional treatment planning information selected by the user may be integrated into the treatment plan as one treatment action to be performed and be optionally also associated with an order for its execution. In other word, the user may determine a treatment plan with one or more actions to be performed in an order that he/she selected. In one example, the probe may be mounted on a robot (i.e., mechanical system) and the treatment plan may be used to control said robot to execute the treatment action(s) under the supervision of the user.

In one embodiment, the device is configured to provide as output, for example visual output, said treatment plan.

FIG. 4 is an example of flow chart of the method for helping the user 4 in the planification of the treatment to be delivered via the probe to the effective target region of the body of the patient.

In a step 100, the three-dimensional scene is displayed in the display unit 9 of the user graphical interface 2. The three-dimensional scene comprises the body 3D model 3 and the probe 3D representation 5.

In a step 200, a current position and current orientation of the probe 3D representation 5 in the three-dimensional scene coordinate system is computed based on a current position and spatial orientation of the controller 6 in the predetermined user coordinate system 12.

In a step 300, an initial treatment 3D distribution 7 corresponding to said current position and current orientation is computed.

In a step 400, a representation of the initial treatment 3D distribution 7 is displayed in the display unit 9, overlaid with the body 3D model 3.

In some embodiments, the user graphical interface 2 comprises croppers.

Via the croppers, the user 4 may crop the body 3D model relative to a 2D plane in the scene coordinate system, so that a 2D cross section of the body 3D model, referred to as body 2D cross section, is displayed on the visualization unit. For instance, the body 2D cross section results from an intersection between the body 3D model and the 2D plane.

Alternatively, via the croppers, the user 4 may crop, relative to a 2D plane in the scene coordinate system, the representation of the treatment 3D distribution computed for a given position and orientation of the probe 3D representation in the scene coordinate system, so that a 2D cross section of the treatment 3D distribution, referred to as treatment 2D cross section, is displayed on the display unit 9. For instance, the treatment 2D cross section results from an intersection between the treatment 3D distribution 7 and the 2D plane.

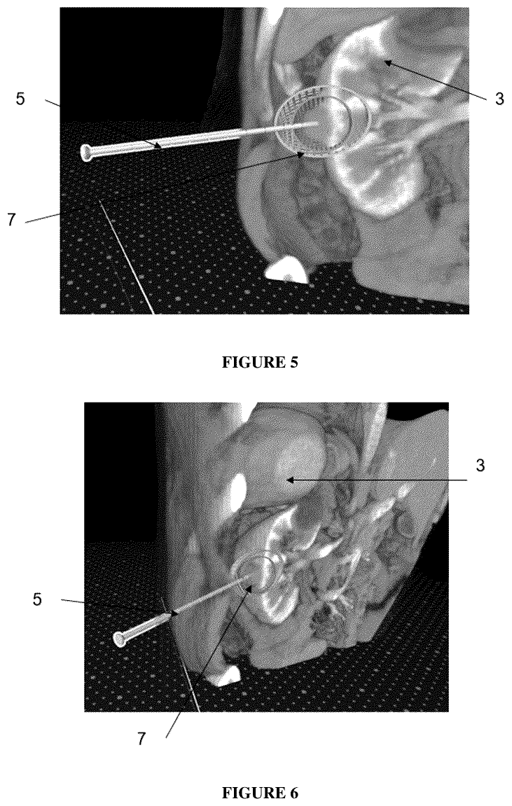

FIGS. 5 and 6 are two examples of a treatment 2D cross section, for different 2D planes.

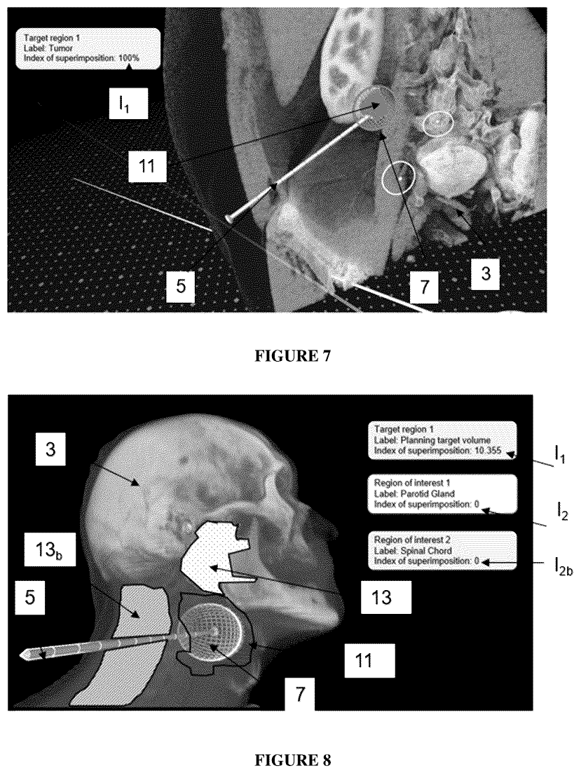

In some embodiments, the three-dimensional scene further comprises a target region 11. The target region 11 is a digital representation of the effective target region of the body of the patient to which the treatment needs to be delivered. The target region 11 may be representative of a region that needs to be reached or hit by the treatment while manipulating the probe during the intervention. For instance, the target region 11 is a tumor to reduce or eliminate. Such a target region 11 may be visible on the body 3D model 3, when the body 3D model 3 has been computed from preoperative images. FIG. 7 shows an example of the three-dimensional scene comprising the target region 11. A label of the target region 11 can be displayed. On FIG. 7, the target region 11 is a tumor.

When the three-dimensional scene comprises such a target region 11, the method for helping the planification of the treatment further includes a step of identifying voxels in the three-dimensional scene, referred to as target voxels, that are both located in the target region and belong to the set of treatment voxels, computed for a given position and orientation of the probe 3D representation 5 in the scene coordinate system. In other words, the step of identifying allows the user getting an insight about how, for the given position and orientation of the probe 3D representation 5, the treatment 3D distribution 7 overlaps or fills the target region 11.

As an indicator of how the treatment 3D distribution 7 fills the target region, a first index of superimposition I₁ can be computed. The first index of superimposition I₁ can be displayed on the three-dimensional scene, as visible on FIG. 7. When for a given treatment voxel, the voxel set of parameters comprises an intensity value, the first index of superimposition I₁ can be computed based on the intensity values of the identified target voxels. Advantageously, this first index of superimposition I₁ provides a quantitative information on the percentage of the target region that would be affected by the energy of the calculated treatment 3D distribution 7.

In one example, the first index of superimposition I₁ is the sum of the intensity values of the identified target voxels. Alternatively, the first index of superimposition I₁ may be calculated as the sum or the weighted sum of the intensity values of x % lower intensity voxels among the identified interest voxels, x ranging from approximately 1 to 20. This is an important information for the user, notably when cancerous tissues must be eradicated, to ensure that with the currently calculated treatment 3D distribution 7 a minimum amount of energy, sufficient to destroy the target cells, is transferred to the target region.

In another example, the first index of superimposition I₁ is a first weighted sum of the intensity values of the identified target voxels. The first weighted sum can be parameterized by weights, where each weight is associated with one among the identified target voxels and is representative of an absorption coefficient of the associated identified target voxel. This example is for instance relevant when it is desired to take into account the different transfer properties of different tissues in the target region.

In some embodiments, the three-dimensional scene further comprises a region of interest 13. The region of interest 13 may be representative of an organ at risk, and thus needs to be protected from the treatment while manipulating the probe during the intervention. In other words, the region of interest 13 may be representative of areas to be avoided by the treatment. Such a region of interest 13 may be visible on the body 3D model 3, when the body 3D model 3 has been computed from preoperative images.

FIG. 8 is an example of a three-dimensional scene comprising a target region 11 and a region of interest 13. A label of the target region 11 and a label of the region of interest 13 are displayed. On FIG. 8, the target region 11 is a planning target volume and the region of interest 13 is a parotid gland of the patient. The three-dimensional scene may further comprise other regions of interest. On FIG. 8, a second region of interest 13b is visible and a label of the second region of interest 13b is displayed. On FIG. 8, the region of interest is a spinal cord.

When the three-dimensional scene comprises such a region of interest 13, the method for helping the planification of the treatment further includes a step of identifying voxels in the three-dimensional scene, referred to as interest voxels, that are both located in the region of interest and belong to the set of treatment voxels, computed for a given position and orientation of the probe 3D representation 5 in the scene coordinate system. In other words, the step of identifying allows the user getting an insight about how, for the given position and orientation of the probe 3D representation 5, the treatment 3D distribution 7 overlaps or hits the region of interest.

As an indicator of how the treatment 3D distribution hits the region of interest, a second index of superimposition I₂ can be computed. The second index of superimposition I₂ can be displayed on the three-dimensional scene, as visible on FIG. 8. When for a given treatment voxel, the voxel set of parameters comprises an intensity value, the second index of superimposition I₂ can be computed based on the intensity values of the identified interest voxels.

In one example, the second index of superimposition I₂ is the sum of the intensity values of the identified interest voxels. The second index of superimposition I₂ may be calculated as sum or weighted sum of the Y % voxels with the highest intensity values among the identified interest voxels, where Y may range from approximately 1 to 20.

In another example, the second index of superimposition I₂ is a second weighted sum of the intensity values of the identified target voxels. The second weighted sum can be parameterized by weights, where each weight is associated with one among the identified interest voxels and is representative of an absorption coefficient of the associated identified interest voxel. This example is for instance relevant when it is desired to take into account the different absorption (i.e., transfer) properties of different tissues in the region of interest.

When the region of interest is representative of areas to be avoided by the treatment, the second index of superimposition I₂ can be used to adjust the position and orientation of the probe 3D representation 5. For instance, a threshold value can be set as a maximum value that cannot be exceed. When the second index of superimposition I₂ is higher than the threshold value, an alert message can be displayed. Alternatively, the display of the region of interest may be modified to alert the user that the overlap between the treatment, for the current position and orientation of the probe, is excessive and might even be dangerous. For instance, the threshold value is inputted by the user through the user graphical interface.

In some embodiments, as already mentioned, the three-dimensional scene comprises both a target region 11 and a region of interest 13. Therefore, both the first index of superimposition I₁ and the second index of superimposition I₂ can be displayed, as illustrated on FIG. 8.

When the three-dimensional scene comprises other regions of interest, such as on FIG. 8, the corresponding second indexes of superimposition can be displayed. On FIG. 8, another second index of superimposition I₂b is displayed and corresponds to the second region of interest 13b.

The method of the present invention may be configured to follow the movement of the controller 6, held by the user, in a continuous manner. As consequence, the current position and spatial orientation of the controller 6 are continuously received and used to update in real time the probe 3D representation 5 in the virtual environment and as well calculate and display in real time the corresponding treatment 3D dose distribution.

In some embodiments, the user can interact with the user graphical interface 2 through the controller 6, in order to, starting from a starting position and a starting orientation of the probe 3D representation, modify the position and orientation of the probe 3D representation 5, and, as a consequence, to modify the treatment 3D distribution 7. Therefore, in those embodiments, the method further comprises:
receiving another current position of the controller 6 in the predetermined user coordinate system, following an interaction of the user with the controller 6,
computing a corrective position and a corrective orientation of the probe 3D representation 5 in the three-dimensional scene coordinate system based on said another current position,
computing a corrective treatment 3D distribution corresponding to the corrective position and corrective orientation,
displaying a representation of the corrective treatment 3D distribution overlaid with said body 3D model.

The corrective treatment 3D distribution corresponds to a new set of voxels, the voxels in the new set of voxels being referred to as corrective treatment voxels.

When the three-dimensional scene comprises a target region 11, the method for helping the planification of the treatment further includes a step of identifying voxels in the three-dimensional scene, referred to as corrective target voxels, that are both located in the target region 11 and belong to the set of corrective treatment voxels, computed for the corrective position and corrective orientation of the probe 3D representation 5 in the scene coordinate system.

As an indicator, the first index of superimposition I₁ is updated, now referred to as first corrective index of superimposition, and computed in a similar way to the first index of superimposition previously described. The first corrective index of superimposition can be displayed on the display unit 9.

When the three-dimensional scene comprises a region of interest 13, the method for helping the planification of the treatment further includes a step of identifying voxels in the three-dimensional scene, referred to as corrective interest voxels, that are both located in the region of interest 13 and belong to the set of corrective treatment voxels, computed for the corrective position and corrective orientation of the probe 3D representation 5 in the scene coordinate system.

As an indicator, the second index of superimposition I₂ is updated, now referred to as second corrective index of superimposition, and computed in a similar way to the second index of superimposition previously described. The second corrective index of superimposition can be displayed on the display unit 9.

Advantageously, a convenient position and orientation of the probe 3D representation 5 can be found when interacting with the user graphical interface 2. Such a convenient position and orientation corresponds for instance to a maximum value of the first index of superimposition I₁ when the three-dimensional scene comprises a target region 11. Therefore, in some embodiments, the method for helping the user in the planification of the treatment to be delivered via the probe further includes computing angular coordinates representative of an orientation of the probe 3D representation 5 with respect to a reference orientation of said body 3D model 3. The angular coordinates will thus be usable to reproduce and reposition the real probe during the real medical intervention aiming at delivering the treatment to the effective target region of the body of the patient in the same manner as during the interaction with the user graphical interface 2. Conveniently, the angular coordinates can be displayed on the display unit 9 and recorded into an external memory in view of the real medical intervention. The computed angular coordinates may be transferred to a mechanical system, mounting the probe, to be used to assist the user in the positioning of the probe during the treatment

Sometimes, it may occur that one unique probe is not sufficient to deliver enough treatment to the target region. In this case, using a second probe can be envisaged.

Therefore, in some embodiments, the three-dimensional scene further comprises at least one other probe 3D representation, representative of at least a portion of another probe. The at least one other probe representation is, like the probe 3D representation 5, interactively controlled by the controller 6. For instance, the number of probe 3D representations in the three-dimensional scene is N, with N being an integer higher or equal to 2.

In those embodiments, each probe 3D representation might be manipulated and moved to a current position and a current orientation, which are recorded. A corresponding individual treatment 3D distribution corresponds to each of the current position and current orientation. A complete treatment 3D distribution can then be computed based on each corresponding individual treatment 3D distribution.

For instance, each individual treatment 3D distribution corresponds to an individual set of voxels, referred to as treatment voxels. Each treatment voxel is associated with an intensity value. The complete treatment 3D distribution can then correspond to a set of voxels, referred to as complete set of treatment voxels, where each voxel in the complete set of treatment voxels is associated with a total intensity value. For example, the total intensity value is computed as a sum of the intensity values of each individual treatment 3D distribution at this voxel.

In those embodiments, when the three-dimensional scene comprises a target region 11, the method for helping the planification of the treatment further includes a step of identifying voxels in the three-dimensional scene, referred to as first complete voxels, that are both located in the target region 11 and belong to the complete set of treatment voxels.

As an indicator, the first index of superimposition I₁ is updated, now referred to as first complete index of superimposition, and computed in a similar way to the first index of superimposition I₁ previously described. The first complete index of superimposition can be displayed on the display unit 9 of the user graphical interface 2.

In those embodiments, when the three-dimensional scene comprises a region of interest 13, the method for helping the planification of the treatment further includes a step of identifying voxels in the three-dimensional scene, referred to as second complete voxels, that are both located in the region of interest 13 and belong to the complete set of treatment voxels.

As an indicator, the second index of superimposition I₂ is updated, now referred to as second complete index of superimposition, and computed in a similar way to the second index of superimposition I₂ previously described. The second complete index of superimposition can be displayed on the user graphical interface 2.

A person skilled in the art will readily appreciate that various embodiments disclosed may be combined without departing from the scope of the invention. Of course, the present invention is not limited to the embodiments described above as examples. It extends to other variants.

1. A computer-implemented method for helping a user in the planification of a treatment to be delivered via a probe to an effective target region of a body of a patient, by means of a user graphical interface comprising a controller configured to control the interaction between the user and the user graphical interface, said treatment being a radiotherapy, an ablation therapy, a laser-based or an ultrasound-based treatment, said method comprising:
displaying in a display of the user graphical interface a three-dimensional scene comprising a body 3D model of at least a portion of a body of the patient and a probe 3D representation of at least a portion of a probe; said probe 3D representation being interactively controlled by the controller; wherein the three-dimensional scene has a corresponding scene coordinate system attached thereto;
computing a current position and current orientation of the probe 3D representation in the said scene coordinate system based on a current position and current orientation of the controller in a predetermined user coordinate system;
computing an initial treatment 3D distribution corresponding to said current position and current orientation of the probe 3D representation;
displaying in the display unit a graphical representation of said initial treatment 3D distribution overlaid with said body 3D model.

2. The method according to claim 1, further comprising cropping said representation, so as to display a 2D cross-section of said initial treatment 3D distribution, in response to an instruction provided by the user via said controller.

3. The method according to claim 1, wherein the initial treatment 3D distribution comprises a set of treatment voxels, each voxel in said set of treatment voxels being associated to a voxel set of parameters.

4. The method according to claim 3, wherein for each voxel in the set of treatment voxels, the associated voxel set of parameters comprises an intensity value.

5. The method according to claim 1, wherein the three-dimensional scene further comprises a target region and/or a region of interest.

6. The method according to claim 5, further comprising identifying target voxels in the set of treatment voxels that are located inside the target region, and/or identifying interest voxels in the set of treatment voxels that are located inside the region of interest.

7. The method according to claim 6, further comprising computing a first index of superimposition based on the sets of parameters of the identified target voxels and displaying said first index of superimposition and/or computing a second index of superimposition based on the sets of parameters of the identified interest voxels and displaying said second index of superimposition.

8. The method according to any one of claim 1, wherein, when the three-dimensional scene further comprises the region of interest, said region of interest is an organ at risk.

9. The method according to claim 1, wherein said method further comprises:
receiving another current position and another current orientation of the controller in the predetermined user coordinate system,
computing a corrective position and a corrective orientation of the probe 3D representation in the scene coordinate system based on said another current position and another current orientation,
computing a corrective treatment 3D distribution corresponding to the corrective position and corrective orientation,
displaying a representation of the corrective treatment 3D distribution overlaid with said body 3D model.

10. The method according to claim 9, wherein said corrective treatment 3D distribution comprises a set of corrective treatment voxels, the method further comprising:
identifying corrective target voxels in the set of corrective treatment voxels that are located inside the target region,
computing a first corrective index of superimposition based on the intensity values of the identified corrective target voxels,
displaying said first corrective index of superimposition.

11. The method according to claim 5, wherein the target region is a tumor.

12. The method according to claim 1, further comprising computing angular coordinates representative of an orientation of the probe 3D representation with respect to a reference orientation of said body 3D model.

13. The method according to claim 1, wherein the computed angular coordinates are transferred to a mechanical system, mounting the probe, to be used to assist the user in the positioning of the probe during the treatment.

14. The method according to claim 1, wherein the three-dimensional scene further comprises at least one other probe 3D representation of a portion of another probe, said at least one other probe 3D representation being interactively controlled by the controller and independently from the probe 3D representation,
said method further comprising:
computing a complete treatment 3D distribution based on contributions of the probe 3D representation and the at least one other probe 3D representation,
displaying a representation of the complete treatment 3D distribution overlaid with said body 3D model.

15. The method according to claim 1, wherein the three-dimensional scene further comprises at least one other probe 3D representation of a portion of another probe, said at least one other probe 3D representation being interactively controlled by the controller and independently from the probe 3D representation,
said method further comprising:
receiving a selection of a subset among the probe 3D representation and the at least one other probe 3D representation in an active state,
computing a complete treatment 3D distribution based on contributions of the received selection,
displaying a representation of the complete treatment 3D distribution overlaid with said body 3D model.

16. The method according to claim 1, wherein the user graphical interface comprises a stereoscopic displaying unit.

17. A device comprising a processor, a user graphical interface comprising a controller configured to control the interaction between a user and the user graphical interface, said processor being configured to carry out a method according to claim 1.

18. A non-transitory program storage device, readable by a computer, tangibly embodying a program of instructions executable by the computer to perform the method of claim 1.
